(19) United States
(12) Patent Application Publication
(10) Pub. No.: US 2016/0370356 A1
(43) Pub. Date: Dec. 22, 2016

(54) PAPER-BASED IMMUNOASSAY WITH POLYMERIZATION-BASED SIGNAL AMPLIFICATION

(71) Applicants: PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Abraham Kwame BADU-TAWIAH, Columbus, OH (US); Mohammad Hussein AL-SAYAH, Sharjah (AE); Kaja KAASTRUP, Lakewood, CO (US); Shefali LATHWAL, Cambridge, MA (US); Dionysios CHRISTODOULEAS, Cambridge, MA (US); Hadley SIKES, Arlington, MA (US); George M. WHITESIDES, Newton, MA (US)

(21) Appl. No.: 15/120,965

(22) PCT Filed: Feb. 24, 2015
(86) PCT No.: PCT/US15/17312

§ 371 Date: Aug. 23, 2016

Related U.S. Application Data
(60) Provisional application No. 61/943,607, filed on Feb. 24, 2014.

(51) Int. Cl. G01N 33/542; G01N 33/569; G01N 33/543; G01N 33/548

(52) U.S. Cl. CPC G01N 33/542; G01N 33/548; G01N 33/56905; G01N 33/56911; G01N 33/54393; G01N 2333/445; G01N 2333/23

(57) ABSTRACT

Disclosed herein are paper-based immunoassays useful for the detection of analytes, kits for the same and methods of using the same.

### RELATED APPLICATIONS

This application claims priority to U.S. Patent Application No. 61/943,607, filed on Feb. 24, 2014, the contents of which are incorporated by reference herein in their entirety.

### STATEMENT OF GOVERNMENT SUPPORT

This invention was made with government support under HR0011-12-2-0010 awarded by the Defense Advanced Research Projects Agency (DARPA), an agency of the United States Department of Defense. The government has certain rights in the invention.

### INCORPORATION BY REFERENCE

The patent and scientific literature referred to herein establishes knowledge that is available to those of skill in the art. The issued U.S. patents, allowed applications, published foreign applications, and references, that are cited herein are hereby incorporated by reference to the same extent as if each was specifically and individually indicated to be incorporated by reference.

### TECHNICAL FIELD

The field of this application generally relates to the detection of analytes using paper-based immunoassays.

### BACKGROUND

Rapid, inexpensive, simple, fieldable tests have become one of the major diagnostic and analytical requirements for disease control. These tests use passive chips or hand-held devices for use by technically unskilled personnel (e.g., famers, law enforcement officers, first responders, and military personnel) in resource limited settings. Diagnostics for civil response to an emerging biological incident—especially one requiring large-scale anticipation and management, large amounts of data, and very large numbers of tests delivered rapidly—requires diagnostic and bioanalytical systems that have the lowest practical cost, function without supporting equipment, and are portable and easy to operate. Ideal diagnostic devices should also require minimal sample handling before analysis, provide time-insensitive results, measure multiple analytes simultaneously, and enable immediate decision-making.

Tropical and zoonosis diseases are of particular interest because they occur in the world's most deprived areas, and could be sources for bioterrorist attack in regions where the disease is not endemic.

For example, malaria is endemic in over 100 countries, and kills approximately 780,000 people per year; 65% of these fatalities are children under the age of 15, predominantly in sub-Sahara Africa. Approximately 40% of the world's population is at risk for contracting Malaria. Among the four human-specific plasmodium species, Plasmodium falciparum causes the most virulent form of human malaria, resulting in up to 300-500 million infections annually.

Basic tests for malaria include subjective diagnosis, where a health care professional uses the patient's history of subjective fever as the indication. However, 30% of patients will no longer have a fever upon arriving at a health care facility, resulting in many false negatives. Microscopic examination of blood films are an inexpensive alternative, but accuracy depends on the skill of the diagnostician being able to determine whether the thickness of the blood is indicative of a malarial infection. Molecular methods using polymerase chain reaction (PCR) are also available, but these are expensive.

In addition to Malaria, brucellosis is another disease of special interest. Brucellosis is the most common zoonosis disease (i.e. transmissible from animals to human), and a significant cause of reproductive losses in animals. In humans, brucellosis is a chronic disease, which can affect a variety of organs. Humans could be infected through direct contact with the tissues of infected animal or by ingestion of contaminated food, water or aerosol. Foodborne transmission is the major source of infection, with unpasteurized raw milk presenting the highest risk.

Clinical diagnosis of brucellosis is not easily achieved. Laboratory testing is based on isolation and characterization of the Brucella bacteria, which is time-consuming and hazardous. The use of molecular methods via PCR-based assays is common, and has enabled rapid recognition and identification of different species and strains but the technique uses expensive equipment, and can hardly be employed in resource-limited settings.

Immunoassay-based diagnostic tests for many diseases (such as malaria, brucellosis, HIV, syphilis, etc.) generally use a two-step process. First, a sample from a patient suspected of having a particular disease is contacted with a substrate having affinity for a component of the disease in a first capture step. Second, reagents are applied to the substrate to produce a signal in a second detection step. Previous immunoassay-based diagnostic tests traditionally used enzyme-linked antibodies in the detection step. However, because enzymes are unstable, the capture and detection steps were required to be done in quick succession.

Two types of rapid diagnostic tests, based on immunoassay, are currently widely used. The difference is mainly found in the mode of signal detection: (a) immunoassays that use enzyme-linked antibodies, and (b) immunoassays that are based on gold-nanoparticle conjugated antibodies. First, immunoassay tests that utilize enzyme-linked antibodies produce a time-dependent signal (i.e. producing a signal which changes over time). These types of rapid diagnostic tests require test results to be recorded after a specific set time. Such operational bottlenecks reduce assay throughput, and may hinder the use of rapid diagnostic tests in mass screening and diagnosis of a specific disease in the event of an outbreak. For this reason, sample-to-sample and patient-to-patient consistency is difficult to achieve. In addition, time periods for the detection step may be extended (e.g., 20 minutes), resulting in an inefficient process, and potentially making consistency more difficult to achieve.

An exemplary schematic for a direct, enzyme-linked immunoassay method is shown in FIG. 5A, wherein a sample containing the antigen of interest (the “analyte”) is immobilized on the support. In some embodiments, the support is then washed to remove unbound antigen and other components of the sample. In some embodiments, a non-reacting component (e.g., bovine serum albumin, casein, or ethanolamine) is then added to block any remaining surface of the support which has not bound the antigen analyte. Next, a primary antibody having affinity for the analyte antigen is added to bind the antigen analyte. In some embodiments, the primary antibody is complexed to an enzyme, and a substrate for the enzyme is then added to produce a signal, indicating the presence of the primary antibody, bound to the antigen analyte, which is in turn immobilized on the support.

An exemplary schematic for an indirect method is shown in FIG. 5B. Indirect methods are similar to direct methods, except the analyte is an antibody (e.g., in the case of brucellosis), and so an antigen having affinity for the primary (analyte) antibody is immobilized on the support. A species-specific secondary antibody which has affinity for the primary antibody is then added. In some embodiments, the secondary antibody is complexed to an enzyme and a substrate for the enzyme is then added to produce a signal, indicating the presence of the secondary antibody bound to the primary antibody, which is in turn bound to the antigen, which is in turn immobilized on the support.

The direct method is problematic in that capture of the antigen analyte to the support (i.e. immobilization) is not specific. Because samples to be tested generally contain other components (e.g., proteins), these other components may be bound to the support, so only a small portion of the antigen of interest is actually bound due to competition with the other components of the sample. Hence, direct immunoassays are typically used for purified antigen analytes. Indirect methods solve this competition problem by first immobilizing an antigen that has affinity for the primary (analyte) antibody so that the primary (analyte) antibody can be captured selectively from all other components (e.g., proteins) in the sample mixture.

An exemplary schematic for a sandwich assay is shown in FIG. 5C. Sandwich assays are used for antigen detection (e.g., in the case of malaria), and they solve the problem of limited antigen binding to the substrate by providing a primary antibody which is a capture antibody having affinity for the antigen of interest (analyte) bound to the support, resulting in a support having affinity for the antigen analyte. The support is then contacted with the sample suspected of containing the antigen of interest. In some embodiments, the support is then washed to remove unbound antigen and other components of the sample. In some embodiments, a non-reacting component (e.g., bovine serum albumin, casein, or ethanolamine) is then added to block any remaining surface of the support which has not bound the capture antibody. Next, a secondary antibody having affinity for the analyte antigen is added to bind the antigen analyte. In some embodiments, the secondary antibody is complexed to an enzyme, and a substrate for the enzyme is then added to produce a signal, indicating the presence of the secondary antibody bound to the antigen analyte, which is in turn bound to the capture antibody, which is in turn immobilized on the support.

The second type of rapid diagnostic tests (aside from the enzyme-linked immunoassays) are based on gold-nanoparticle conjugated antibodies. An exemplary schematic for a sandwich assay using antibodies labeled with gold nanoparticles (“AuNP”) is shown in FIG. 6. AuNP-based rapid diagnostic tests in part eliminate the time-dependency problems of enzyme-linked antibody rapid diagnostic tests. However, AuNP-based immunoassays produce a colorimetric signal due to aggregation of AuNPs, and accordingly do not offer signal amplification during detection and thus have limited sensitivity. AuNP tests result in an immediate color change which is observable by eye. However, the color of the AuNP depends on the concentration of the nanoparticles and their proximity to one another. Because of this proximity requirement (AuNPs need to be approximately 1 micron apart on the support in order to produce an observable color change), AuNP tests have rigorous requirements for types of substrates that may be used. Because substrates such as ordinary chromatography or filter paper, cellulose and other porous polymer fabrics have large pore sizes (on the order of about 10 to about 50 microns, and corresponding to about 60% void space in paper), it is unsuitable for use in a AuNP test. In addition, the gold components required for use in the tests are also expensive.

Overall, antigen-based immunoassays, however, are promising diagnostic tools for malaria detection—especially in resource-limited settings—because they require no sample pretreatment, and can be used by unskilled personnel. Over 20 antigen-based immunoassays are commercially available today (e.g., those based on antigens such as Plasmodium Glutamate dehydrogenase (pGluDH), Histidine-rich protein II (HRP II), P. falciparum lactate dehydrogenase (pLDH)), but all are expensive compared to blood film analysis.

Polymerization-based amplification experiments have previously been reported, but are limited to functional glass substrates, polystyrene, or in a microfluidic format, all requiring a staining step to visualize a hydrogel/polymer formed as a result of molecular recognition. See Sikes, H. D. et al. Nature Mat. 2008, 7, 52-56; Berron, B. J. et al. Lab Chip 2012, 12, 708; Berron, B. J. et al. Biotech. Bioeng. 2011, 108(7), 1521-1528. However, staining on a porous substrate such as paper can stain the entire surface, resulting in a false positive.

A need exists for rapid, time-independent and inexpensive diagnosis of diseases such as malaria, brucellosis, HIV, and West Nile Virus in settings extending from diagnosis of imported malaria in tertiary hospitals in regions where the disease is not endemic to remote health care clinics lacking laboratories.

### SUMMARY

Paper-based diagnostic devices, kits and methods for the detection of analytes are described. See generally Badu-Tawiah, A. K. et al. Lab Chip 2015, 15, 655-659, the contents of which are incorporated herein by reference in their entirety.

Disclosed herein are low-cost and easy-to-use paper-based rapid diagnostic tests which decouple infectious antigen/antibody detection from signal amplification in a time-independent manner. The disclosed rapid diagnostic tests, use readily available and inexpensive materials (e.g., paper) and reagents (e.g., stable organic compounds, antibodies) to develop an immunoassay for the detection of analytes.

In one aspect, disclosed herein is a method of detecting an analyte of interest in a sample, the method comprising providing a paper support; contacting said paper support with a sample, said paper support capturing at least a portion of any analyte present in said sample; contacting said paper support with a first antibody, wherein said first antibody has affinity for and binds to said analyte; and wherein said first antibody comprises a polymerization catalyst; contacting said paper support with a monomer composition, wherein said monomer composition comprises a monomer component capable of being polymerized in the presence of said polymerization catalyst, wherein at least a portion of said monomer component forms a polymer; and detecting said polymer, wherein detecting the presence of said polymer indicates presence of said analyte.

In another aspect, disclosed herein is a kit for detecting an analyte of interest in a sample, the kit comprising a paper support capable of capturing an analyte; a first antibody for binding said analyte, wherein said first antibody has affinity for said analyte, and wherein said first antibody comprises a polymerization catalyst; a monomer composition, wherein said monomer composition comprises a monomer component capable of being polymerized in the presence of said polymerization catalyst.

In another aspect, disclosed herein is a kit for detecting an analyte of interest in a sample, the kit comprising a paper support comprising a capture antibody or antigen coupled to said paper support, wherein said capture antibody or antigen has affinity for said analyte; a second antibody for binding said analyte, wherein said second antibody has affinity for said analyte, and wherein said second antibody comprises a polymerization catalyst; and a monomer composition, wherein said monomer composition comprises a monomer component capable of being polymerized in the presence of said polymerization catalyst.

In another aspect, disclosed herein is a method of making a paper support for detecting an analyte, the method comprising providing a paper support; contacting said paper support with an oxidizing agent to produce an aldehyde-functionalized paper support; contacting said aldehyde-functionalized paper support with said capture antibody/antigen, thereby covalently bonding said capture antibody/antigen to said paper support, wherein said capture antibody/antigen has affinity for said analyte.

In some embodiments, the disclosed rapid diagnostic tests comprise (1) a paper support, (2) an antibody functionalized with a polymerization catalyst, (3) a monomer composition capable of being polymerized in the presence of said polymerization catalyst, and (4) a polymerization initiator. In an exemplary rapid diagnostic test, a sample suspected of containing an analyte of interest is contacted either directly with the paper support (e.g., in a direct method) or to a paper support functionalized with an antigen having affinity for the primary (analyte) antibody (e.g., in an indirect method) or to a capture antibody having affinity for the antigen analyte (e.g., in a sandwich method) to immobilize at least a portion of the analyte, and unbound sample is removed by washing. A functionalized antibody having affinity for the analyte of interest is then contacted with the resulting support, and excess functionalized antibody is removed by washing. The support is then treated with a monomer composition, and an initiator is introduced to induce polymerization via the polymerization catalyst. Polymerization results in hydrogel formation only in the areas of the support comprising bound analyte due to the fact that the polymerization catalyst is only present in these areas of the support due to the selective binding of the functionalized antibody to these areas. Unpolymerized monomer composition is removed by washing, and the analyte of interest can be detected by observing the areas of the support which comprise hydrogel, either directly (e.g., via a color change in the polymerized monomer composition in a colorimetric method) or indirectly (e.g., by various chemical, electrical, or spectroscopic methods well-known in the art, such as staining, scanning, fluorescence, UV absorption, magnetism, etc.).

The disclosed rapid diagnostic tests provide a number of advantages over prior rapid diagnostic tests. The disclosed rapid diagnostic tests allow for the diagnosis of a wide variety of diseases, enable mass screening by a limited number of health professionals, point of care testing, self-testing by patients at home (which can allow for signal development and analysis later, upon arrival at a health care facility).

In comparison to colorimetric methods that are currently used with paper-based immunoassays, the disclosed rapid diagnostic tests require ten-fold less time for the signal amplification and visualization steps—2-2.5 minutes compared to 20-30 minutes for enzymatic and AuNP-based methods. This reduction in time combined with the ability to stop, store and restart the disclosed paper-based assay has the potential to minimize false readouts due to time constraints in situations where only a few health workers are tending to the needs of many patients. The high visual contrast provided by the disclosed tests, even close to the limit of detection, also makes it easier for a user to visually interpret the results, in comparison to enzymatic and AuNP-based methods where low contrast can lead to ambiguous visual interpretation.

Further, the disclosed rapid diagnostic tests are paper-based, and paper provides a number of advantages over supports used in prior assays. For example, paper is commercially available, fabricated on a large-scale all over the world, is widely abundant, inexpensive, biodegradable, renewable and allow for one-step functionalization (e.g., by periodate oxidation to form aldehyde-functionalized paper in wet solution or gas-phase silanization). The disclosed rapid diagnostic tests are also energy efficient, not requiring the use of pumps for liquids, as liquid wetting of the various components utilized is driven by capillary action. The disclosed rapid diagnostic tests do not require staining, instead allowing detection of analytes by more direct methods (e.g., direct visualization without the use of a stain). Because the support is paper, washing of the support is rapid and effective due to the large pore size of paper as compared to other supports, such as nylon membrane with smaller pores. The rapid diagnostic tests are flexible, allowing detection of both antigens (e.g., in direct, or sandwich methods) and antibodies (e.g., in indirect methods) as the analyte. Because of this flexibility, the disclosed diagnostics allow for detection of antigen or antibody analytes associated with any disease for which an antibody or antigen analyte is known (e.g., malaria, brucellosis, HIV, West Nile Virus, etc.).

In some embodiments, the disclosed rapid diagnostic tests include eosin as the polymerization catalyst and a tertiary amine co-initiator. Although eosin is oxygen-sensitive, the conditions and time scales of the disclosed rapid diagnostic tests overcome oxygen inhibition, allow detection in an ambient environment. See Kaastrup, K.; Sikes, D. H. Lab Chip 2012, 12, 4055-4058. This is particularly useful in non-laboratory settings. The disclosed rapid diagnostic tests are specific (avoiding false positive results), sensitive (avoiding false negative results), user-friendly (simple to perform, using specimens obtained by non-invasive means), rapid, and deliverable (readily accessible to end-users). The disclosed rapid diagnostic tests are low cost, fast, time-independent, sensitive and consistent. In some embodiments, the disclosed rapid diagnostic tests are estimated to cost $0.50 USD per test, representing approximately more than a 30% reduction in total cost over rapid diagnostic tests which use a membrane such as Immunodyne.

### BRIEF DESCRIPTION OF THE DRAWINGS

The following figures are provided for the purpose of illustration only and are not intended to be limiting.

FIGS. 1A-B show the results of colorimetric detection of molecular recognition after amplification via photo polymerization in an exemplary biotin amine-functionalized oligonucleotide/eosin-functionalized streptavidin system using direct methodology. FIG. 1A shows an exemplary positive result, with the paper support demonstrating a visible pink color (shown as a dark circle in the center of FIG. 1A). FIG. 1B shows an exemplary negative result, with no apparent pink color visible on the paper support (i.e. no dark circle visible).

FIGS. 2A-B show the results of colorimetric detection of molecular recognition after amplification via photo polymerization at 90 seconds. FIG. 2A shows an exemplary negative result, with no apparent pink color visible on the paper support. FIG. 2B shows an exemplary positive result, with the paper support demonstrating a visible pink color (shown as a dark circle in the center of FIG. 2B).

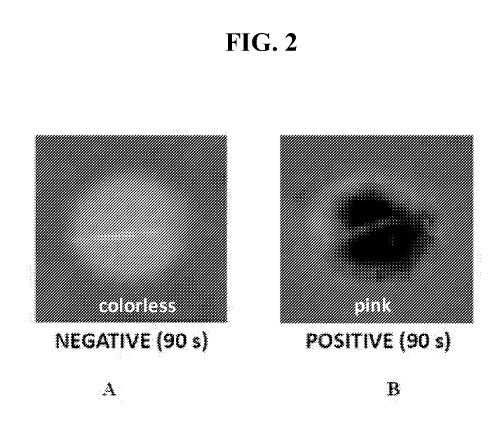

FIGS. 3A-B show the results of colorimetric detection of molecular recognition after amplification via photo polymerization in the exemplary biotin amine-functionalized oligonucleotide/eosin-functionalized streptavidin system of Example 2. Biotinylated amine functionalized oligonucleotides (A) and amine functionalized oligonucleotides (B) (1 μL, of 100 μM solutions) were immobilized on aldehyde functionalized chromatography No. 1 paper. In both sample (A) and control (B), 10 μL, of eosin-functionalized streptavidin (10 μg/mL) was added to the test zone, in a humid chamber for 5 minutes after which it was washed three times. Polymerization was achieved by adding a phenolphthalein-doped monomer composition and applying light (wavelength 522 nm) at different set times. Binding-responsive polymers were visualized by applying 4-6 μL, of 0.01 M NaOH solution to the test zone. FIG. 3A shows an exemplary positive result, with the paper support demonstrating a visible pink color (shown as a dark circle in the center of FIG. 3A) after 90 and 100 seconds. FIG. 3B shows an exemplary negative result, with no observable pink color at 100, 110 and 120 seconds.

FIGS. 4A-B show the results of colorimetric detection of HRP-2 antigen after amplification via photo polymerization in an exemplary system. FIG. 4A shows a negative control using PBS buffer without HRP-2 antigen. FIG. 4B shows a positive control (shown as a dark circle in the center of FIG. 4B) using 10 μg/mL of HRP-2 antigen in PBS buffer. In both negative and positive controls, 1 μL, of 2.9 mg/mL of capture antibody was first immobilized in the test zones, after which the test zone was blocked. Then, 10 μL, of PBSA (1% BSA in 1×PBS) buffer with and without the HRP-2 antigen were added to the test zones, respectively for positive and negative controls, in a humid chamber for 15 minutes. In both negative and positive controls, 5 μL, of eosin-functionalized reporter antibody were added and allowed to stand for another 15 minutes after which test zone was washed three times. Polymerization was achieved by adding a phenolphthalein-doped monomer composition and applying light (wavelength 522 nm) for 70 seconds. Binding-responsive polymers were visualized by applying 2 μL, of 0.5 M NaOH solution to the test zone. FIG. 4A shows an exemplary negative result, with no apparent pink color visible on the paper support at 70 seconds. FIG. 4B shows an exemplary negative result (shown as a dark circle in the center of FIG. 4B), with the paper support demonstrating a visible pink color at 70 seconds. For the purposes of comparison, detection of HRP-2 antigen before amplification via fluorescence is shown in FIGS. 4C-D.

FIGS. 5A-C show schematic of exemplary embodiments of direct, indirect and sandwich enzyme-linked immunoassays assays for antigen and antibody screening. FIG. 5A shows a schematic of a direct antigen screening assay whereby the analyte antigen is immobilized on the paper support. A functionalized antibody is bound to the analyte, and a substrate for the enzyme is added to produce a detectable signal. FIG. 5B shows a schematic of an indirect antibody screening assay whereby the an antigen is immobilized on the paper support and the analyte antibody is bound to the antigen. A functionalized secondary antibody is then bound to the analyte antigen, and a substrate for the enzyme is added to produce a detectable signal. FIG. 5C shows a schematic of a sandwich antigen screening assay whereby a capture antibody is immobilized on the paper support. An analyte antigen is bound to the capture antibody and a functionalized secondary antibody is bound to the analyte, and a substrate for the enzyme is added to produce a detectable signal.

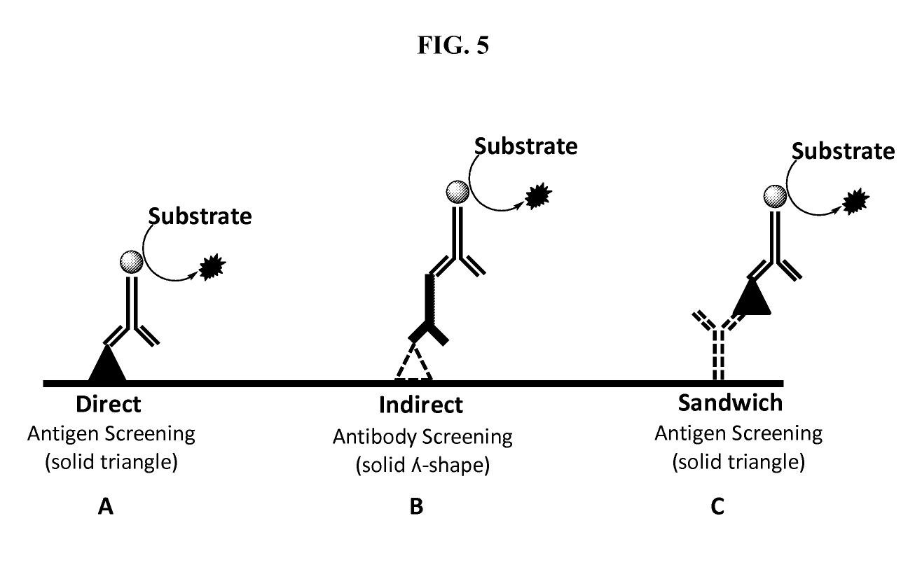

FIG. 6 shows a schematic for a traditional sandwich assay based on gold nanoparticles (“AuNPs”). A capture antibody is immobilized on a nitrocellulose support and the analyte parasite is bound to the capture antibody. A secondary antibody functionalized with a gold nanoparticle is bound to the analyte. Detection of the gold nanoparticles requires proximity of one gold nanoparticle to another, producing a visible color change.

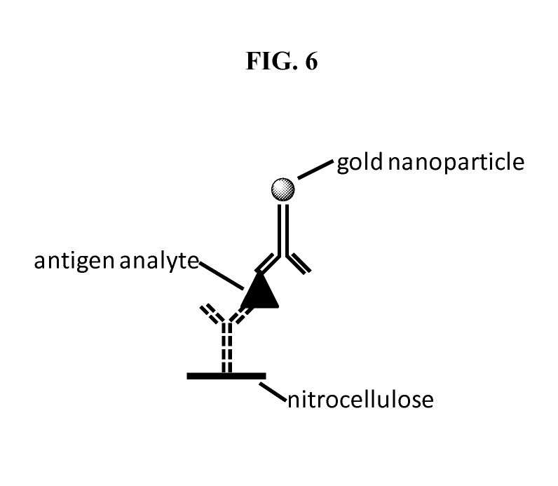

FIGS. 7A-C show exemplary schematics of direct, indirect and sandwich polymer-amplified immunoassays for antigen and antibody screening according to the present disclosure, whereby a capture antibody/antigen is immobilized on a paper support. FIG. 7A shows a direct method, wherein the antigen analyte is immobilized on the paper support and the paper support is subsequently treated with a primary antibody functionalized with a polymerization catalyst. The primary antibody has affinity for and binds the antigen analyte and thereby becomes immobilized on the paper support through the antigen analyte. The paper support is then contacted with a monomer composition and exposed to a polymerization initiator, which initiates polymerization of the monomer composition on the areas of the paper support in proximity to the primary antibody functionalized with the polymerization catalyst. FIG. 7B shows an indirect method used to detect an antibody analyte. An antigen having affinity for the primary (analyte) antibody is immobilized on the paper support. A species-specific secondary antibody having affinity for the primary (analyte) antibody is coupled to a polymerization catalyst. Accordingly, the antigen has affinity for and binds the primary (analyte) antibody, and the secondary antibody has affinity for and binds the primary antibody, both of which become immobilized on the paper support, the primary antibody immobilized through the antigen, and the secondary antibody immobilized through the primary antibody, which is in turn immobilized through the antigen. The paper support is then contacted with a monomer composition and exposed to a polymerization initiator, which initiates polymerization of the monomer composition on the areas of the paper support in proximity to the secondary antibody functionalized with the polymerization catalyst. FIG. 7C shows a sandwich method where a capture antibody is bound to the paper support. The antigen analyte is then immobilized on the paper support through the capture antibody, and the paper support is subsequently treated with a secondary antibody functionalized with a polymerization catalyst. The secondary antibody has affinity for and binds the antigen analyte, becoming immobilized on the paper support through the antigen analyte and the capture antibody. The paper support is then contacted with a monomer composition and exposed to a polymerization initiator, which initiates polymerization of the monomer composition on the areas of the paper support in proximity to the secondary antibody functionalized with the polymerization catalyst.

FIG. 8 shows the structure of eosin Y, and shows a schematic of eosin activation initiating polymerization of a monomer composition using triethanolamine as a co-initiator to result in polymer formation.

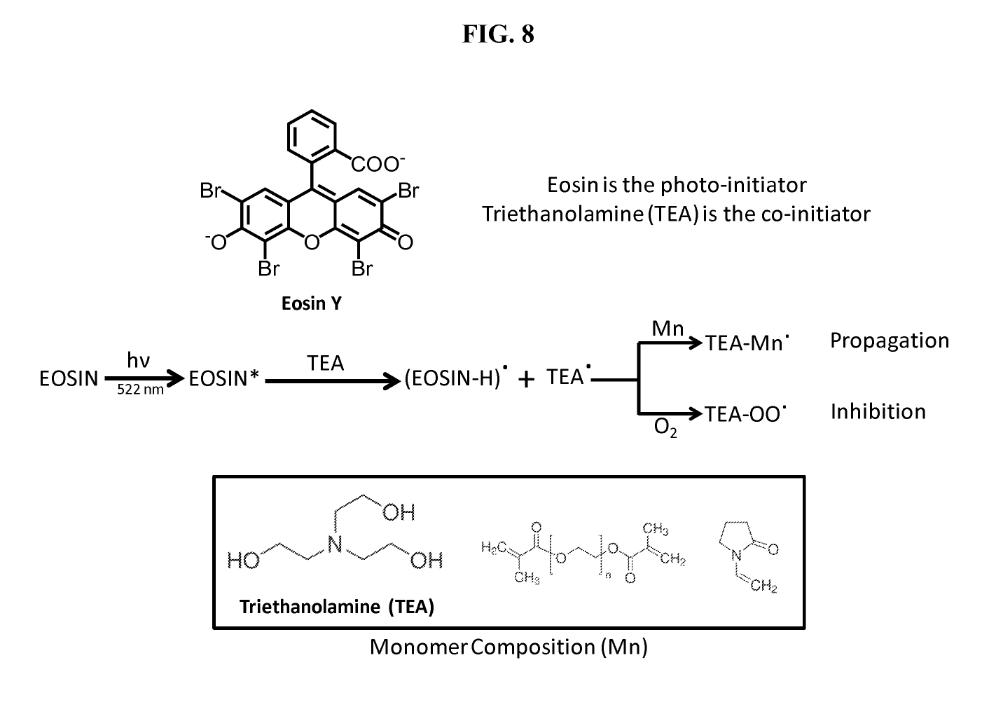

FIG. 9 shows a schematic of eosin activation initiating polymerization of a monomer composition using triethanolamine as a co-initiator to result in polymer formation.

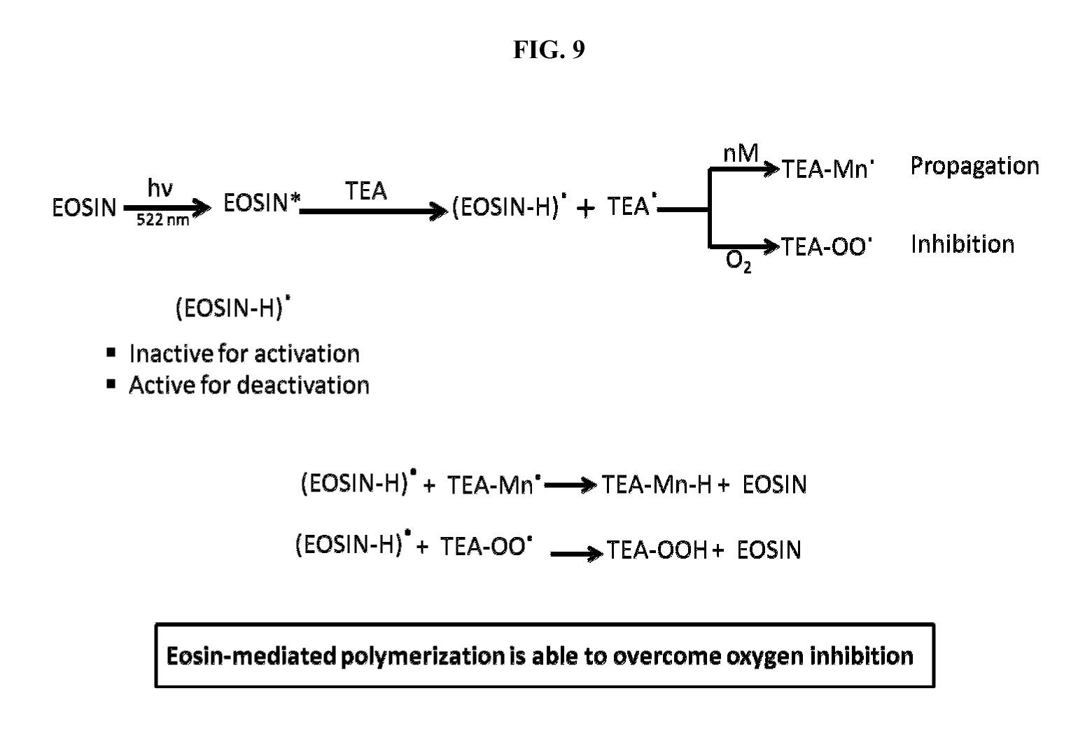

FIGS. 10A-B show schematics of traditional (“old”) format polymer-based antigen screening as compared to the rapid diagnostic tests disclosed herein. FIG. 10A shows a prior sandwich-type assay which requires staining to visualize detection of the antigen analyte. FIG. 10B shows a sandwich-type assay in accordance with the present disclosure which does not require staining to visualize detection of the antigen analyte due to the phenolphthalein-doped monomer composition.

FIG. 11A shows an exemplary schematic for functionalization of a paper support using 0.03 M potassium periodate at 65° C. for two hours. FIG. 11B shows an exemplary demonstration of treatment of untreated and aldehyde-functionalized paper with 2,4-dinitrophenylhydrazone, where untreated paper turns yellow, but aldehyde-functionalized paper reacts with 2,4-dinitrophenylhydrazone to give a deep-brown color. FIG. 11C shows a schematic of the reaction of 2,4-dinitrophenylhydrazone with a carbonyl moiety of the functionalized paper.

FIG. 12 shows the equilibrium between colorless and pink isomers of phenolphthalein as a function of pH.

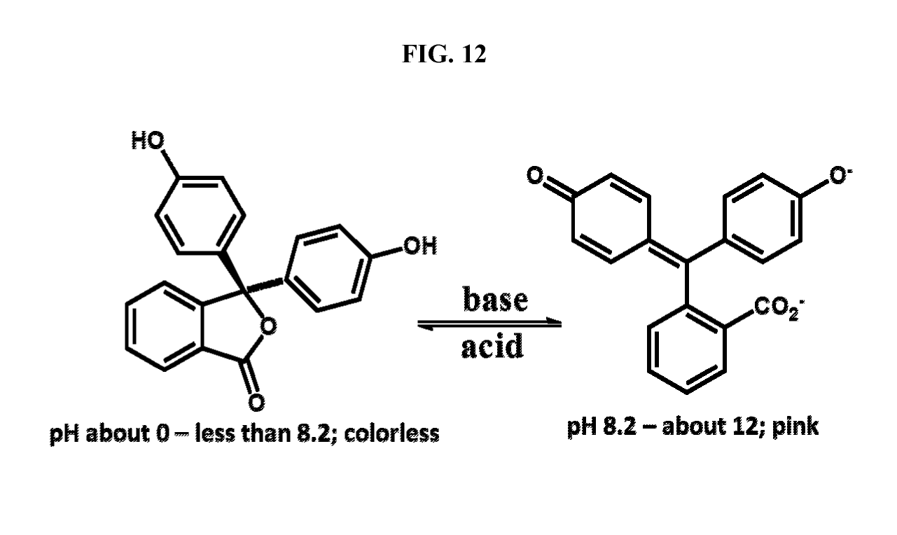

### DETAILED DESCRIPTION

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

Disclosed herein is a colorimetric method that integrates a paper-based immunoassay with a rapid, visible-light-induced polymerization to provide high visual contrast between a positive and a negative result.

The rapid diagnostic tests disclosed herein are useful for detecting a wide array of analytes and diseases. In some embodiments, the analyte is an oligonucleotide. In some embodiments, the analyte is an antibody. In some embodiments, the analyte is an antigen. In some embodiments, the analyte is plasmodium falciparum histidine-rich protein 2 (HRP-2). In some embodiments, the analyte is ABMAL-0444. In some embodiments, the analyte is ABMAL-0445. In some embodiments, the analyte is the p24 antigen. In some embodiments, the analyte is Plasmodium Glutamate dehydrogenase (pGluDH). In some embodiments, the analyte is P. falciparum lactate dehydrogenase (pLDH). In some embodiments, the analyte is IgG and IgM antibodies released from Brucella Abortus infection. In some embodiments, the analyte is human IgG4.

Without wishing to be bound by theory, the inventors believe signal amplification is achieved in the disclosed rapid diagnostic tests due to the presence of multiple initiator molecules localized at or near the paper surface for analyte binding event. Each initiator molecule can initiate polymerization, leading to growth of polymer chains and amplification of the signal.

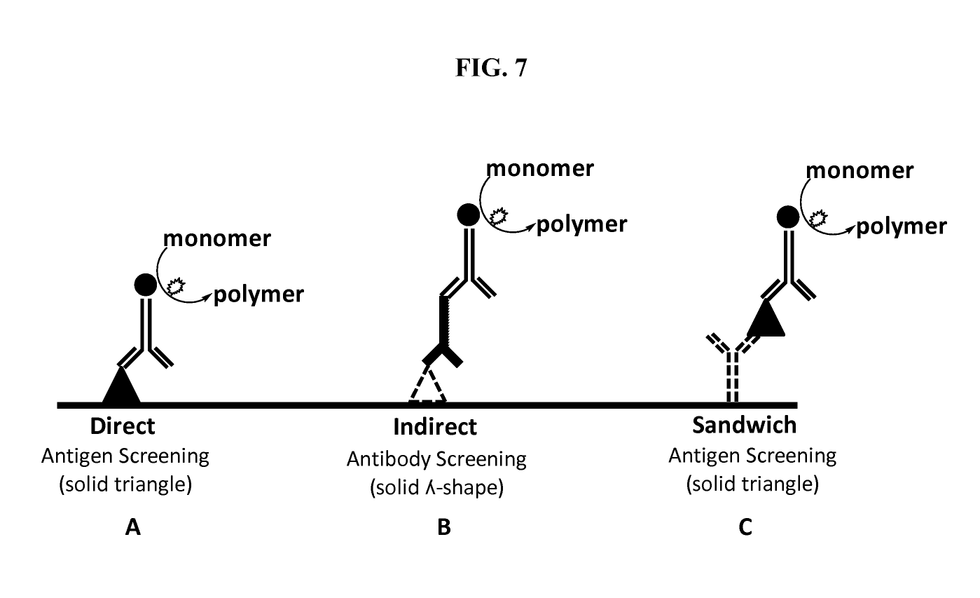

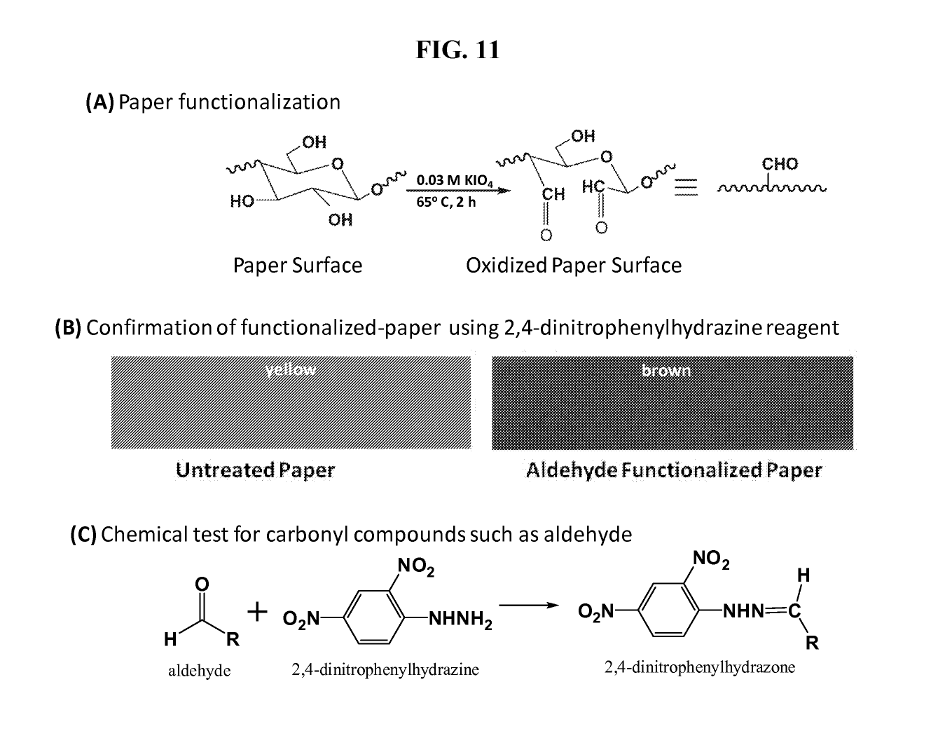

FIGS. 7A-C show exemplary schematics of direct, indirect and sandwich assays according to the present disclosure. The schematics shown in FIGS. 7A-C show paper supports. Paper supports useful in the disclosed assays include all types of cellulose materials that allow printing of wax-demarcated test zones. Wax printing requires two steps and produces hydrophobic barriers (for the test zones) that extend through the thickness of the paper. After wax printing, the paper is heated, and the wax melts and spreads vertically into the paper, creating the hydrophobic barrier needed to confine test reagents. Examples of paper supports used in this invention include Whatman™ filter papers, chromatography papers, polymeric-based membranes, and cotton or nylon fabric. In some embodiments, the paper support is functionalized by oxidizing the surface with an oxidation agent to provide aldehyde-functionalized paper for antigen/antibody immobilization, as shown in FIG. 11. In some embodiments, the paper is coated with agarose, which is then oxidized to provide the aldehyde functionalities useful for antigen/antibody immobilization. In some embodiments, the paper is coated with chitosan, which is then reacted with glutaraldehyde to provide the aldehyde functionalities useful for antigen/antibody immobilization. In some embodiments, multiple layers of antigen/antibody are prepared on the paper by alternatively adding antigen/antibody and glutaraldehyde on the paper. In some embodiments, the first layer of antigen/antibody is formed by using the original aldehyde functionalities present on the paper; followed by treatment with glutaraldehyde, which anchors the second layer to the first via cross-linking. In some embodiments, the exposed aldehyde functionalities are then reacted with an antigen or antibody to covalently bond these components to the paper support. In some embodiments, the unreacted aldehyde moieties are then blocked by treating the paper support with a non-reacting component (e.g., bovine serum albumin, casein, or ethanolamine) to provide a stable paper support ready to be shipped or used immediately in a diagnostic test.

The schematics shown in FIGS. 7A-C also show functionalized antibodies. A functionalized antibody is an antibody with affinity for an analyte or another antibody which is functionalized with and coupled to a polymerization catalyst. The schematics shown in FIGS. 7A-C also show polymerization catalysts, monomer compositions and polymerization initiators.

In the direct method shown in FIG. 7A, the antigen analyte is immobilized on the paper support and the paper support is subsequently treated with a primary antibody functionalized with a polymerization catalyst. In some embodiments, the antigen analyte is present in a sample suspected of containing the antigen analyte, and the sample is contacted with the paper support. The primary antibody has affinity for and binds the antigen analyte and thereby becomes immobilized on the paper support through the antigen analyte. The paper support is then contacted with a monomer composition and exposed to a polymerization initiator, which initiates polymerization of the monomer composition on the areas of the paper support in proximity to the primary antibody functionalized with the polymerization catalyst. Unreacted monomer composition may then be washed away, leaving polymer only on areas of the paper support in proximity to the primary antibody and the antigen analyte. Presence of the polymer, indicating the presence of the analyte, is then detected. Exemplary detection methods include, but are not limited to, direct visual observation, colorimetric readout, staining, pH change, scanning, and spectroscopic methods such as fluorescence, UV absorption or transmission.

The indirect method shown in FIG. 7B is similar to the direct method shown in FIG. 7A, except it is used to detect an antibody analyte. An antigen having affinity for the primary (analyte) antibody is immobilized on the paper support. A species-specific secondary antibody having affinity for the primary (analyte) antibody is coupled to a polymerization catalyst. Accordingly, the antigen has affinity for and binds the primary (analyte) antibody, and the secondary antibody has affinity for and binds the primary antibody, both of which become immobilized on the paper support, the primary antibody immobilized through the antigen, and the secondary antibody immobilized through the primary antibody, which is in turn immobilized through the antigen. Accordingly, in some embodiments, the analyte is present in a sample suspected of containing the analyte, and the sample is contacted with the paper support. As in the schematic of the direct method shown in FIG. 7A, the paper support is then contacted with a monomer composition and exposed to a polymerization initiator, which initiates polymerization of the monomer composition on the areas of the paper support in proximity to the secondary antibody functionalized with the polymerization catalyst. Unreacted monomer composition may then be washed away, leaving polymer only on areas of the paper support in proximity to the secondary antibody, primary antibody, and the antigen. Presence of the polymer, indicating the presence of the analyte, is then detected. Exemplary detection methods are as disclosed above with respect to the direct method schematic shown in FIG. 7A.

The sandwich method shown in FIG. 7C is similar to the direct and indirect methods shown in FIGS. 7A-B, except a capture antibody is bound to the paper support in place of the antigen. The antigen analyte is then immobilized on the paper support through the capture antibody, and the paper support is subsequently treated with a secondary antibody functionalized with a polymerization catalyst. In some embodiments, the antigen analyte is present in a sample suspected of containing the antigen analyte, and the sample is contacted with the paper support (which comprises the capture antibody). The secondary antibody has affinity for and binds the antigen analyte, becoming immobilized on the paper support through the antigen analyte and the capture antibody. As in the schematic of the direct method shown in FIG. 7A, the paper support is then contacted with a monomer composition and exposed to a polymerization initiator, which initiates polymerization of the monomer composition on the areas of the paper support in proximity to the secondary antibody functionalized with the polymerization catalyst. Unreacted monomer composition may then be washed away, leaving polymer only on areas of the paper support in proximity to the secondary antibody, antigen analyte and capture antibody. Presence of the polymer, indicating the presence of the analyte, is then detected. Exemplary detection methods are as disclosed above with respect to the direct method schematic shown in FIG. 7A.

The resultant polymer in turn becomes immobilized to the paper support, and can clearly be distinguished from polymers formed in bulk solution, which are easily washed away. Without wishing to be bound by theory, it is postulated that reaction of immobilized/activated radicals with radical species of the polymer in a termination step is responsible for the polymer immobilization phenomenon. Other mechanism of polymer immobilization may involve some physical interactions between the polymer and the proteins on the surface, or interaction with paper support. In addition, in some embodiments the polymer is not soluble in water and so after attached to the surface it cannot be washed away. In some embodiments, the polymer forms a hydrogel.

The disclosed rapid diagnostic tests demonstrate improved stability over enzyme-based methods due to the lack of unstable enzymes. This allows for signal amplification by polymerization to be conducted either immediately after capturing the antigen/antibody or at a later time, without affecting the diagnosis outcome. Notably, a typical rapid diagnostic test according to the present disclosure is time-independent at a number of stages, providing for a flexible diagnostic method which can be readily prepared, shipped, stored and testing procedures which can be flexibly conducted without rigid adherence to time limits or storage conditions. For example, an exemplary step-wise procedure for manufacturing an exemplary rapid diagnostic test according to the present disclosure is depicted below:
1) React paper support with oxidizing agent to provide aldehyde-functionalized paper.
2) Immobilize capture antibody on aldehyde-functionalized paper.
3) Block unreacted aldehyde sites with non-reacting component.
4) Treat paper support with sample suspected of containing analyte of interest.
5) Wash paper support to remove unbound analyte.
6) Treat paper support with functionalized antibody.
7) Wash paper support to remove unbound functionalized antibody.
8) Treat paper support with monomer composition which optionally comprises a colorimetric indicator.
9) Expose paper support to stimulus to polymerize monomer composition in areas containing functionalized antibody bound to analyte.
10) Wash to remove unpolymerized monomer composition.
11) Detect formation of the polymer formed in areas containing functionalized antibody bound to analyte, optionally by treatment to develop the colorimetric indicator.

As discussed herein, the items listed above can be categorized into three separate steps: (a) support preparation, steps 1-3; (b) analyte capture, steps 4-7; and (c) analyte detection, steps 8-11. After step 3 (block unreacted aldehyde sites with non-reacting component), a paper is produced which can be stored and shipped (for example, as part of a kit). The assay process can also be stopped indefinitely without risk of degradation of the components of the test after step 7 (wash paper support to remove unbound functionalized antibody). Further, in certain embodiments, the polymerization reaction is largely time-independent (i.e. the polymerization can precisely be turned “on” and “off” with the stimulus), meaning the time after which step 11 is carried out (i.e. detect formation of the polymer formed in areas containing functionalized antibody bound to analyte) is not critical to the results of the test. Notably, in some embodiments, eosin molecules immobilized on a support are capable of initiating polymerization after four months, six months, or more. In some embodiments, the time of the detection process (i.e. the initiation step) is short (about 60 seconds), in contrast with the time scale on the order of minutes for enzyme-based immunoassays. In some embodiments, the initiation step itself can be performed in less than 35 seconds. In some embodiments, the detection step can be effectively terminated (i.e. turned “on” or “off”) by removing the light source, something which is not easily achieved in enzyme-linked immunoassays. In some embodiments, the development of the color used as readout is not dependent on the time between the taking of sample and initiating the assay; in other words, the color produced is stable with time. In some embodiments, the color produced is stable for several months. In some embodiments, a protective layer is applied to the paper to prolong the stability of the color produced. In some embodiments, the protective layer comprises tape.

In some embodiments, step 11 referenced above is achieved by adding phenolphthalein to the monomer composition. This assay mode is particularly useful under resource-limited settings, with no need for staining, scanning, or the use of spectroscopic methods. Phenolphthalein is colorless at a pH range of about 0 to less than 8.2, and does not affect the polymerization. Upon polymerization (step 9), the indicator is trapped in the polymer which in turn is immobilized on the paper support. Its color changes to pink upon the addition of a basic solution (for example, about 2 to about 6 μL of about 0.01 to about 0.51 M NaOH), thus providing a visual photometric detection of the polymer, which in turn indicate the presence of analyte.

In developing the disclosed tests, the inventors surprisingly discovered that when using a colorimetric indicator (such as phenolphthalein, naphtholphthalein, o-cresolphthalein, and thymolphthalein), polymerization reactions did not occur as efficiently with a colored monomer composition as compared to a colorless monomer composition. Based on the prior art, it was believed that a basic pH would be required, which in the case of phenolphthalein would result in a colored monomer composition. See, e.g., Cruise, G. M. et al. Biotechnol. Bioeng. 1998, 57(6), 655-665. Instead, basic pH and colored monomer compositions did not efficiently produce interfacial polymerizations, instead producing little or no polymerization (i.e. a false negative) or bulk polymerization (i.e. a false positive). For example, in one instance, the inventors found that the initial color of the monomer composition comprising phenolphthalein was pink due to the presence of the basic triethanolamine co-initiator, but the polymerization reaction did not efficiently produce interfacial polymerizations, instead producing little or no polymerization (i.e. a false negative) or bulk polymerization (i.e. a false positive). The inventors were surprisingly able to solve this problem by acidifying the monomer composition to a pH at which the colorimetric indicator was colorless, which permitted efficient binding-responsive interfacial polymerization, and subsequent adjustment of the pH post-polymerization to provide the desired colorimetric indication of polymer formation.

The antibody-based immunoassays disclosed herein are also promising diagnostic tools for brucellosis detection in the field. For example, farmers will be able to screen large population of animals, and to diagnose and identify infected animals by themselves in their own farms. Unlike other diseases, brucellosis may require multiplexed detection of both IgG and IgM antibodies. In this case, paper provides unique opportunity for multiplexing since on-chip splitting of sample fluid using 3D-paper-based microfluidic device is straightforward, with no active pumping (see Martinez, A. W. et. al. Proc. Natl. Acad. Sci. USA, 2008, 105, 19606-19611), something which is not easily achieved on other supports such as nitrocellulose or nylon membranes.

In one aspect, a method of detecting an analyte of interest in a sample is disclosed, the method comprising (a) providing a paper support; (b) contacting the paper support with a sample, the paper support capturing at least a portion of any analyte present in the sample; (c) contacting the paper support with a first antibody; wherein the first antibody has affinity for and binds to the analyte; and wherein the first antibody comprises a polymerization catalyst; (d) contacting the paper support with a monomer composition; wherein the monomer composition comprises a monomer component capable of being polymerized in the presence of the polymerization catalyst; wherein at least a portion of the monomer component forms a polymer in the presence of the polymerization catalyst, resulting in a polymer; and wherein detecting the presence of the polymer indicates presence of the analyte.

In some embodiments, the method further comprises the step of (e) applying a polymerization initiator to the paper support, initiating polymerization in the presence of the polymerization catalyst.

In some embodiments, the method further comprises the step of (f) removing unpolymerized monomer composition from the paper support by washing with a first liquid. In some embodiments, the first liquid is deionized water.

In some embodiments, the monomer composition is adjusted or buffered to an appropriate pH. In some embodiments where the detection step requires a specific pH range, the monomer composition is adjusted or buffered appropriately to ensure this pH range is not reached until detection is desired. In some embodiments, the monomer composition comprises phenolphthalein, and the pH of the monomer composition is adjusted or buffered using an acid prior to the detection step. In some embodiments, the acid is hydrochloric acid.

In some embodiments, the paper support directly captures at least a portion of any analyte present in the sample.

In some embodiments, the paper has affinity for the analyte.

In some embodiments, the paper is not nitrocellulose.

In some embodiments, the paper support is covalently bound to a capture antibody or antigen which has affinity for the analyte.

In some embodiments, the capture antibody or antigen is covalently bound to the paper support by reacting the capture antibody or antigen with an aldehyde-functionalized paper to produce the paper support.

In some embodiments, the capture antibody or antigen is covalently bound to the paper support by reacting the capture antibody or antigen with an aldehyde-functionalized paper, followed by blocking unreacted aldehydes to produce the paper support. In some embodiments, the unreacted aldehydes are blocked with an agent selected from at least one of bovine serum albumin, casein and ethanolamine.

In some embodiments, the analyte is selected from an antigen and an antibody.

In some embodiments, the polymerization catalyst comprises a photoinitiator. In some embodiments, the photoinitiator is selected from the group consisting of at least one of AIBN (azobisisobutyronitrile), benzoyl peroxide, DMPA (2,2-dimethoxy-2-phneylacetophenone), acryloyl chloride, NO₂ and peroxides.

In some embodiments, the polymerization catalyst comprises at least one of eosin, methylene blue, and ketocoumarin.

In some embodiments, the polymerization catalyst comprises at least one of triethanolamine, triethylamine, and N-methyldiethanolamine.

In some embodiments, the polymerization catalyst comprises at least a co-initiator. In some embodiments, the co-initiator comprises at least one of triethanolamine, triethylamine, and N-methyldiethanolamine.

In some embodiments, the polymerization initiator is selected from the group consisting of at least one of light, heat, cooling, application of a magnetic field, application of an electrical field, application of electrical current, a chemical reagent and electricity. In some embodiments, the polymerization initiator is light. In some embodiments, the light comprises light having a wavelength of about 522 nm. In some embodiments, the polymerization initiator is light and the light is applied by way of a light box. In some embodiments, the light box comprises a timer. In some embodiments, the light source is an array of light-emitting diodes (“LEDs”) with pulsing light at about 522 nm (about 30 mW/cm²). In some embodiments, the light box applies light from above the paper support. In some embodiments, the light is produced by an array of light-emitting diodes (LEDs).

In some embodiments, the monomer composition comprises poly(ethylene glycol) diacrylate, N-vinylpyrrolidone, triethanolamine, eosin, PEGDA (polyethylene glycol diacrylate), TPT (trimethylolpropane triacrylate), or mixtures thereof.

In some embodiments, the detecting is colorimetric.

In some embodiments, quantitative information regarding analyte levels is obtained. In some embodiments, quantitative information regarding analyte levels is obtained by imaging. In some embodiments, the imaging is computer-based. In some embodiments, the imaging is cellphone-based. In some embodiments, the images are analyzed with software. In some embodiments, the software is NIH's “ImageJ.” In some embodiments, a light stage is used to enhance reproducibility and reliability of the colorimetric readout.

In some embodiments, the monomer composition further comprises an indicator. In some embodiments, the indicator is at least one of pH-sensitive, light-sensitive, temperature-sensitive, sensitive to electrical field or current, or sensitive to magnetic field. In some embodiments, the indicator comprises phenolphthalein and the method further comprises the step of treating the paper support with a base prior to detecting formation of the polymer. In some embodiments, the indicator comprises phenolphthalein.

In some embodiments, the detecting formation of the polymer comprises observing a color change mediated by phenolphthalein under basic conditions.

In some embodiments, the sample comprises blood, plasma, serum, or urine. In some embodiments, the sample comprises human serum.

In another aspect, a kit for detecting an analyte of interest in a sample is disclosed, the kit comprising (a) a paper support capable of capturing an analyte; (b) a first antibody for binding the analyte; wherein the first antibody has affinity for the analyte; and wherein the first antibody comprises a polymerization catalyst; (c) a monomer composition; wherein the monomer composition comprises a monomer component capable of being polymerized in the presence of the polymerization catalyst.

In some embodiments, the kit further comprises instructions for use of the kit for detecting an analyte in a sample.

In another aspect, a kit for detecting an analyte of interest in a sample is disclosed, the kit comprising (a) a paper support comprising a capture antibody or antigen coupled to the paper support; wherein the capture antibody or antigen has affinity for the analyte; (b) a second antibody for binding the analyte; wherein the second antibody has affinity for the analyte; and wherein the second antibody comprises a polymerization catalyst; and (c) a monomer composition; wherein the monomer composition comprises a monomer component capable of being polymerized in the presence of the polymerization catalyst.

In some embodiments, the kit further comprises instructions for use of the kit for detecting an analyte in a sample.

In some embodiments, the application of a polymerization initiator to the paper support causes at least a portion of the monomer component to form a polymer.

In some embodiments, the paper support is covalently bound to the capture antibody or antigen.

In some embodiments, the capture antibody or antigen is covalently bound to the paper support by reacting the capture antibody or antigen with an aldehyde-functionalized paper to produce the paper support.

In some embodiments, the capture antibody or antigen is covalently bound to the paper support by reacting the capture antibody or antigen with an aldehyde-functionalized paper, followed by blocking unreacted aldehydes to produce the paper support. In some embodiments, the unreacted aldehydes are blocked with an agent selected from at least one of bovine serum albumin, casein and ethanolamine.

In some embodiments, the analyte is selected from an antigen and an antibody.

In some embodiments, the polymerization catalyst comprises a photoinitiator. In some embodiments, the photoinitiator is selected from the group consisting of at least one of AIBN (azobisisobutyronitrile), benzoyl peroxide, DMPA (2,2-dimethoxy-2-phneylacetophenone), acryloyl chloride, NO₂ and peroxides.

In some embodiments, the polymerization catalyst comprises at least one of eosin, methylene blue, and ketocoumarin.

In some embodiments, the polymerization catalyst comprises at least one of triethanolamine, triethylamine, and N-methyldiethanolamine.

In some embodiments, the polymerization catalyst comprises at least a co-initiator. In some embodiments, the co-initiator comprises at least one of triethanolamine, triethylamine, and N-methyldiethanolamine.

In some embodiments, the polymerization initiator is selected form the group consisting of at least one of light, heat, cooling, application of a magnetic field, application of an electrical field, application of electrical current, a chemical reagent and electricity. In some embodiments, the polymerization initiator is light. In some embodiments, the light comprises light having a wavelength of about 522 nm. In some embodiments, the polymerization initiator is light and the light is applied from above by way of a light box. In some embodiments, the light box comprises a timer.

In some embodiments, the monomer composition comprises poly(ethylene glycol) diacrylate, N-vinylpyrrolidone, triethanolamine, eosin, PEGDA (polyethylene glycol diacrylate), TPT (trimethylolpropane triacrylate), or mixtures thereof.

In some embodiments, the detecting is colorimetric.

In some embodiments, the monomer composition further comprises an indicator. In some embodiments, the indicator comprises at least one of phenolphthalein, naphtholphthalein, o-cresolphthalein, or thymolphthalein. In some embodiments, the indicator comprises phenolphthalein.

In some embodiments, the paper support is treated with a base prior to detecting formation of the polymer. In some embodiments, the formation of the polymer comprises observing a color change mediated by phenolphthalein under basic conditions.

In some embodiments, the sample comprises blood, plasma, serum, or urine.

In another aspect, a method of making a paper support for detecting an analyte is disclosed, the method comprising (a) providing a paper support; (b) contacting the paper support with an oxidizing agent to produce an aldehyde-functionalized paper support; (c) contacting the aldehyde-functionalized paper support with the capture antibody/antigen, thereby covalently bonding the capture antibody/antigen to the paper support; wherein the capture antibody/antigen has affinity for the analyte.

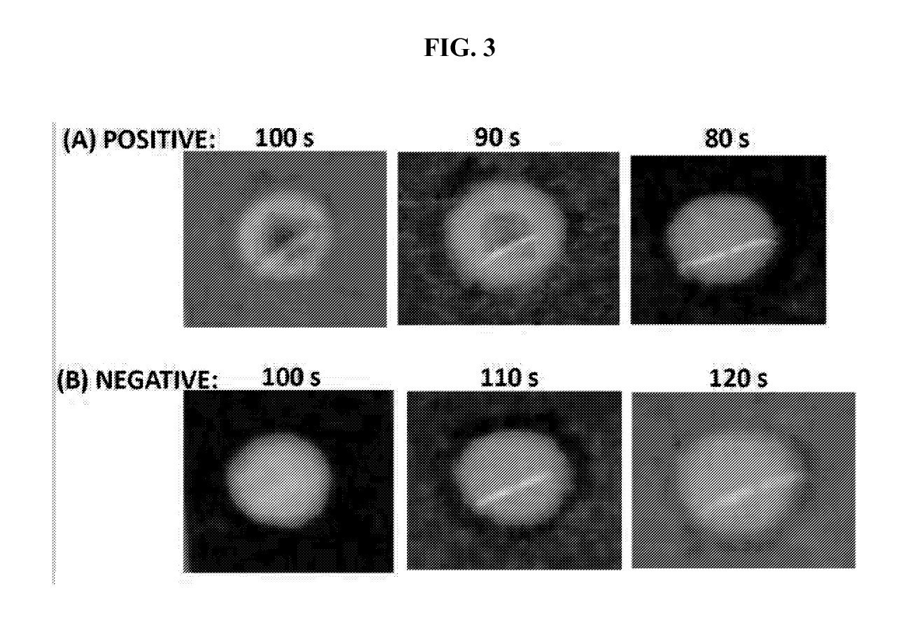

In some embodiments, the polymerization process is photo sensitive. In these embodiments, time-dependent problems associated with enzyme-mediated amplification methods are completely eliminated. As long as the complex formed after the specific binding event is stable, the complex can be amplified and detected at any given time, not necessarily immediately or after a specific duration. The amplification process (i.e. polymerization) itself can be terminated effective simply by removing the light source (compare 90 and 80 seconds in FIG. 3A).

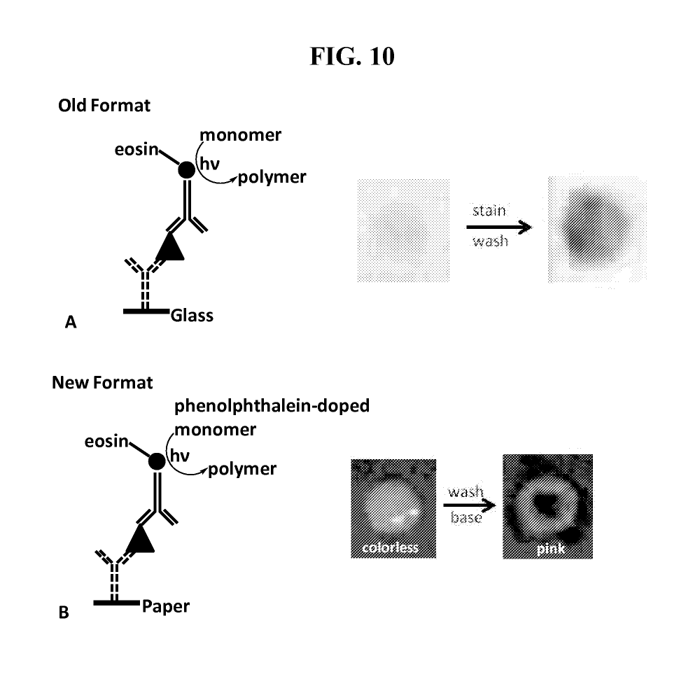

In some embodiments, the disclosed rapid diagnostic tests are colorimetric. In some embodiments, phenolphthalein is used as an indicator. Phenolphthalein is colorless at acidic and slightly basic pH (about 0 to less than 8.2), but turns pink at higher pH (8.2 to about 12). In some embodiments, the monomer composition comprises phenolphthalein (“a phenolphthalein-doped monomer”) such that when the monomer component polymerizes in the presence of the polymerization catalyst on exposure to a polymerization initiator, a hydrogel forms, trapping the phenolphthalein within the hydrogel. Phenolphthalein in unpolymerized monomer composition is washed away where no hydrogel forms. Exposure to basic conditions results in rearrangement of phenolphthalein to a species which displays a pink color, as shown in FIG. 10. The use of a phenolphthalein-doped monomer composition obviates the need for a staining step, which was previously utilized for the visualization of polymer on glass, also eliminating false positive responses produced by staining of the support itself in the absence of a hydrogel.

In some embodiments, an antibody used in the disclosed rapid diagnostic test is selected from the group consisting of ABMAL-0442 Malaria HRPII mAb, ABMAL-0443 Malaria HRPII mAbor, ABMAL-0444, ABMAL-0445, or combinations thereof. In some embodiments, the antibody is anti-IgG. In some embodiments, the antibody is anti-IgM. In some embodiments, an antigen used in the disclosed rapid diagnostic test is Brucella smooth lipopolysaccharide antigen.

In some embodiments, the paper support is functionalized by oxidation prior to contacting with a capture antibody having affinity for an analyte of interest. In some embodiments, the paper support is functionalized by oxidation prior to contacting with an analyte of interest. In some embodiments, the paper is functionalized with a periodate oxidation reagent to form aldehydes. In some embodiments, the periodate oxidation reagent is potassium periodate. In some embodiments, after the aldehyde-functionalized paper support is contacted with a capture antibody or analyte of interest, unreacted aldehyde sites are blocked with a non-reacting component (e.g., bovine serum albumin, casein, or ethanolamine).

In some embodiments, the paper support is first functionalized by oxidation to form an aldehyde-functionalized paper support. The aldehyde-functionalized paper support is then contacted with a capture antibody to immobilize said capture antibody. The paper support is subsequently contacted with a non-reacting component (e.g., bovine serum albumin, casein, or ethanolamine) to block unreacted aldehyde sites. The resulting paper support is then stable and can be readily packaged and shipped, for example as part of a kit.

### DEFINITIONS

As used herein, the term “eosin” refers to eosin Y (also known as eosin Y ws, eosin yellowish, Acid Red 87, C.I. 45380, bromoeosine, bromofluoresceic acid, D&C Red No. 22), eosin B (eosin bluish, Acid Red 91, C.I. 45400, Saffrosine, Eosin Scarlet, or imperial red), or mixtures thereof.

As used herein, the term “capture antibody” refers to an antibody used to immobilize an antigen analyte on a support.

As used herein, the term “functionalized antibody” refers to an antibody coupled to a polymerization catalyst. In some embodiments, the functionalized antibody is directly coupled to a polymerization catalyst. In some embodiments, the functionalized antibody is coupled to biotin, and a polymerization catalyst conjugated to streptavidin which is used to localize the polymerization catalyst to the functionalized antibody. In some embodiments, a functionalized antibody is a primary antibody or a secondary antibody.

As used herein, the term “primary antibody” refers to antibody having affinity for the analyte. In some embodiments, the primary antibody is functionalized. In some embodiments, the primary antibody is not functionalized. As is known in the art, antibodies can be functionalized with a number of components, such as a polymerization catalyst, without substantially affecting their specificity for antigens and other antibodies.

As used herein, the term “secondary antibody” refers to a species-specific antibody having affinity for the primary (analyte) antibody. In some embodiments, the secondary antibody is functionalized. In some embodiments, the secondary antibody is not functionalized.

As used herein, the term “antibody,” including capture antibodies, functionalize antibodies, primary antibodies and secondary antibodies encompasses biotinylated antibodies. In some embodiments which utilize biotinylated antibodies, the support is contacted with streptavidin conjugated to eosin or eosin isothiocyanate (“EITC”) after the support has been contacted with the biotinylated antibody.

As used herein, the term “paper” refers to any porous, hydrophilic media, including any substrate that wicks fluids by capillary action. Exemplary papers include, but are not limited to, chromatographic paper, nitrocellulose, filter paper, cloth, cellulose fabric, and porous polymer film.

As used herein, the term “polymerization catalyst” refers to any catalyst capable of inducing polymerization of a monomer composition. Exemplary polymerization catalysts include, but are not limited to, eosin, methylene blue, and ketocoumarin. In some embodiments, the polymerization catalyst is used in combination with amines as co-initiators.

As used herein, the term “monomer composition” refers to a composition which comprises one or more monomer components capable of being polymerized in the presence of a polymerization catalyst. Exemplary monomer components include, but are not limited to, poly(ethylene glycol) diacrylate, N-vinylpyrrolidone, and triethanolamine.

As used herein, the term “monomer component” refers to a compound capable of being polymerized by a polymerization catalyst. In some embodiments, the monomer component is capable of being polymerized by a polymerization catalyst in the presence of a co-initiator and polymerization initiator. In some embodiments, the monomer component is capable of being polymerized by a polymerization catalyst in the absence of a co-initiator. Exemplary monomer components include, but are not limited to, poly(ethylene glycol) diacrylate, N-vinylpyrrolidone, and triethanolamine.

As used herein, the term “co-initiator” refers to any chemical compound capable of forming a transient proton-bound complex with a polymerization catalyst. Without wishing to be bound by theory, the complex thus formed allows easy abstraction of electron from polymerization catalyst by the co-initiator. The co-initiator becomes a radical, which reacts with the monomer component, thus initiating the polymerization. Exemplary co-initiators include, but are not limited to, triethanolamine, triethylamine, and N-methyldiethanolamine.

As used herein, the term “polymerization initiator” refers to any stimulus capable of inducing polymerization of a monomer composition in the presence of a polymerization catalyst. Exemplary polymerization initiators include, but are not limited to, light, changes in temperature, application of a magnetic or electrical field, application of electrical current, and chemical reagents.

As used herein, the term “affinity” refers to a specific physical and/or chemical force that binds a given antibody to a specific antigen or antibody. In some embodiments, this force can be driven by hydrogen bonding. In some embodiments, the binding force can be driven by electrostatic interactions. In some embodiments, this force can be driven by Van der Waal's interactions. In some embodiments, this force can be driven by shape complementarity. In some embodiments, this force can be driven by hydrophobic or hydrophilic interactions. In some embodiments, this force can be driven by the formation of covalent bonding.

### EXAMPLES

### Example 1

### Preparation of Aldehyde-Functionalized Paper Support

Chromatography No. 1 paper was soaked in 0.031 M KIO₄ solution and heated to 65° C. for two hours. Treated paper was dried under vacuum, in a desiccator. Test zones approximately 2 mm in diameter were created on the dry treated paper by wax printing to create circular hydrophobic barriers on the paper support.

### Example 2

### Proof of Concept with Eosin-Functionalized Streptavidin

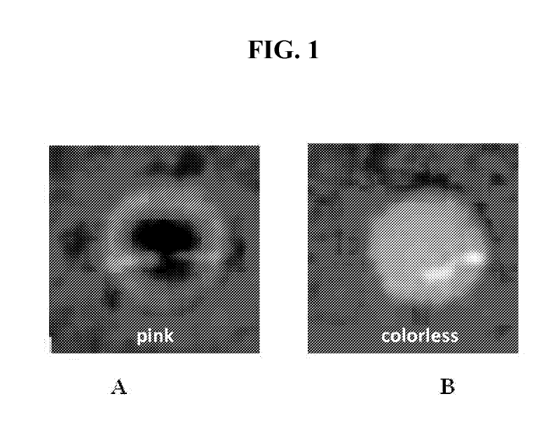

Biotinylated amine-functionalized oligonucleotides were immobilized (10 μL of 100 μM solution) on aldehyde functionalized chromatography No. 1 paper prepared as in Example 1 overnight in a humid chamber. For a negative control, amine-functionalized oligonucleotides (10 μL of 100 μM solution) were immobilized on paper overnight in a humid chamber. After washing away unbound oligonucleotides (2×200 μL 1×PBS), the spot zone was blocked with 10 μL of 1% PBSA (1% BSA in 1×PBS). When the paper was dry, 100 μL of eosin functionalized streptavidin solution (10 μg/mL, made in 0.5% PBSA, 5×Denhardt's solution in 1.5×PBS) was added to the test zone, in a humid chamber for 5 minutes after which it was washed three times using 200 μL each of 0.1% Tween in 1×PBS, 1×PBS, and water. To initiate polymerization, 20 μL of a phenolphthalein-doped monomer composition (200 mM poly(ethylene glycol)diacrylate, 100 mM 1-vinyl-2-pyrrolidone, 150 mM triethanolamine, 0.35 μM eosin and 1.6 mM phenolphthalein in 10% v/v ethanol, adjusted to below pH 8.2 until colorless using 0.02 N hydrochloric acid) was added and light (wavelength 522 nm, intensity 30 mW/cm²) was shone from about 9±1 mm above the paper for 80, 90, 100, 110 or 120 seconds. After washing the test zone with 20 μL of water, binding-responsive hydrogels (pink color) were visualized by adding 4-60 μL of 0.01 M NaOH solution. The results showed that the specific reaction between biotin and streptavidin allowed eosin to be immobilized in the test zones, with pink color seen solely in areas immobilized with biotinylated amine-functionalized oligonucleotides. Although eosin is present in the monomer composition, polymer formation was observed only when there is a high concentration of eosin on the surface (FIG. 1B) provided by immobilization/binding of the functionalized streptavidin. In the presence of specific binding and monomer composition, the amount of hydrogel formed and the amount of trapped phenolphthalein, as indicated by color intensity, is proportional to time of photo-initiation. See FIG. 3A. As a result, higher (pink) color intensity is observed for longer (light) exposure times. No polymer formation (and thus no pink color) were observed in the case of the negative controls because the immobilized oligonucleotides are not biotin functionalized, and hence no molecular recognition occurred. See FIG. 3B. A sample is considered positive at a given polymerization time if for the same or greater time, no polymer forms on the negative control.

### Example 3

### Detection of P. falciparum HRP-2 Antigen with Eosin-Functionalized HRP-2 Specific Secondary Antibodies

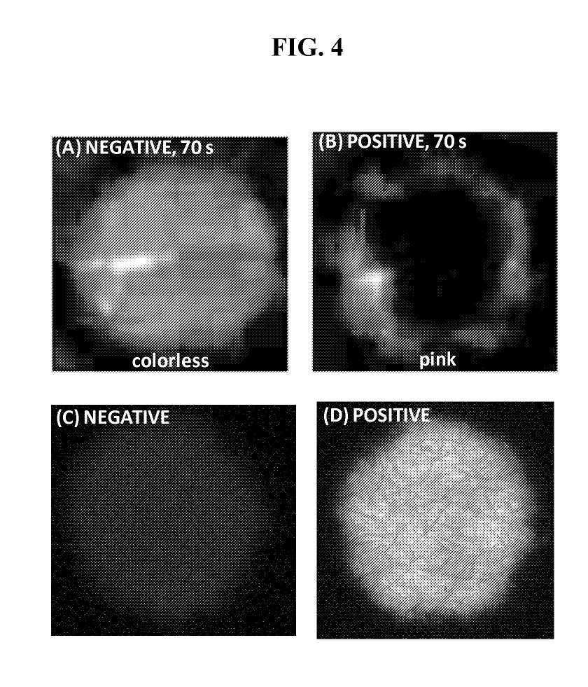

A sandwich immunoassay was used for detection of plasmodium falciparum HRP-2 antigen. About 1 μL of 2.9 mg/mL of a capture antibody specific to HRP-2 was chemically immobilized on aldehyde-functionalized paper, prepared as in Example 1, overnight. Unreacted antibody was washed away (2×20 μL 1×PBS), unreacted sites were blocked (10 μL 1% PBSA for 30 minutes), blocking solution was then washed away (2×20 μL 1×PBS). A sample containing 10 μL of 10 μg/mL of the target HRP-2 was deposited on the paper and incubated for 15 minutes, resulting in binding of the antigen to the capture antibody. After washing unbound sample (2×20 μL 1×PBS), a solution of 5 μL of 50 μg/mL eosin-conjugated secondary antibody was added to the surface and allowed to incubate for 15 minutes. The secondary antibody bound to the antigen and anchored the polymerization catalyst (eosin) to the paper support. Unbound secondary antibody was washed away (20 μL 0.1% Tween 20 in 1×PBS, 20 μL 1×PBS and 20 μL water). Polymerization and visualization steps then proceeded as described for Example 2, except unreacted monomer was washed away with water (20 μL/wash) and visualization was accomplished using 2 μL 0.5 M NaOH. All incubation steps were carried out in a humid chamber. See FIG. 4 for results.

### Example 4

### Detection of Brucella Abortus Antibody with Eosin-Functionalized Mouse-Specific Secondary Antibodies

An indirect immunoassay was used for detection of Brucella IgG antibodies. An antigen (Brucella smooth lipopolysaccharide antigen) specific to Brucella antibody was chemically immobilized on the aldehyde-functionalized paper, which was prepared as in Example 1. A sample containing the target antibody was deposited on the paper, resulting in binding of the antibodies to the immobilized antigen. After washing, a solution of eosin-conjugated secondary antibody was added to the surface. This secondary antibody (i.e. anti-mouse IgG) binds to the analyte antibody and anchors the photo-initiator (eosin) to the paper. Polymerization and visualization steps then proceeded as for Example 2.

Those skilled in the art would readily appreciate that all parameters and configurations described herein are meant to be exemplary and that actual parameters and configurations will depend upon the specific application for which the systems and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. The present invention is directed to each individual feature, system, or method described herein. In addition, any combination of two or more such features, systems or methods, if such features, systems or methods are not mutually inconsistent, is included within the scope of the present invention.

1. A method of detecting an analyte of interest in a sample, the method comprising:
(a) providing a paper support;
(b) contacting said paper support with a sample, said paper support capturing at least a portion of any analyte present in said sample;
(c) contacting said paper support with a first antibody;
wherein said first antibody has affinity for and binds to said analyte; and
wherein said first antibody comprises a polymerization catalyst;
(d) contacting said paper support with a monomer composition;
wherein said monomer composition comprises a monomer component capable of being polymerized in the presence of said polymerization catalyst;
wherein at least a portion of said monomer component forms a polymer; and
(e) detecting said polymer;
wherein detecting the presence of said polymer indicates presence of said analyte.

2. The method of claim 1, wherein applying a polymerization initiator to said paper support initiates said polymerization of at least a portion of said monomer component.

3. The method of claim 1, further comprising the step of:
(f) removing unpolymerized monomer composition from said paper support by washing with a first liquid.

4. The method of claim 1, where said paper support directly captures at least a portion of any analyte present in said sample.

5. The method of claim 4, wherein said paper support has affinity for said analyte.

6. The method of claim 1, wherein said paper support is covalently bound to a capture antibody or antigen which has affinity for said analyte.

7. The method of claim 6, wherein said capture antibody or antigen is covalently bound to said paper support by reacting said capture antibody or antigen with an aldehyde-functionalized paper to produce said paper support.

8. The method of claim 6, wherein said capture antibody or antigen is covalently bound to said paper support by reacting said capture antibody or antigen with an aldehyde-functionalized paper, followed by blocking unreacted aldehydes to produce said paper support.

9. The method of claim 8, wherein said unreacted aldehydes are blocked with an aldehyde blocking agent.

10. The method of claim 1, wherein said analyte is selected from an antigen and an antibody.

11. The method of claim 1, wherein the polymerization catalyst comprises a photoinitiator.

12. The method of claim 1, wherein the polymerization catalyst comprises at least a co-initiator.

13. The method of claim 2, wherein said polymerization initiator is selected from the group consisting of at least one of light, heat, cooling, application of a magnetic field, application of an electrical field, application of electrical current, a chemical reagent and electricity.

14. The method of claim 13, wherein said polymerization initiator is light.

15. The method of claim 1, wherein said monomer composition comprises poly(ethylene glycol) diacrylate, N-vinylpyrrolidone, triethanolamine, or mixtures thereof.

16. The method of claim 1, wherein the detecting is colorimetric.

17. The method of claim 1, wherein said monomer composition further comprises an indicator.

18. The method of claim 17, wherein said indicator comprises phenolphthalein and said method further comprises the step of treating said paper support with a base prior to detecting formation of said polymer.

19. The method of claim 18, wherein detecting formation of said polymer comprises observing a color change mediated by phenolphthalein under basic conditions.

20. The method of claim 17, wherein said indicator comprises phenolphthalein, said monomer composition is at a pH of less than 8.2, and said method further comprises the step of treating said paper support with a base after initiating polymerization and prior to detecting formation of said polymer.

21. The method of claim 20, wherein the pH of the monomer composition is adjusted to less than about 8.2 using an acid.

22. The method of claim 21, wherein the acid is hydrochloric acid.

23. The method of claim 1, wherein said sample comprises blood, plasma, serum, or urine.

24. A kit for detecting an analyte of interest in a sample, the kit comprising:
(a) a paper support capable of capturing an analyte;
(b) a first antibody for binding said analyte;
wherein said first antibody has affinity for said analyte; and
wherein said first antibody comprises a polymerization catalyst;
(c) a monomer composition;
wherein said monomer composition comprises a monomer component capable of being polymerized in the presence of said polymerization catalyst.

25. The kit of claim 24, further comprising a basic aqueous solution.

26. The kit of claim 25, wherein the basic aqueous solution is aqueous sodium hydroxide.

27. The kit of claim 24, further comprising instructions for use of said kit for detecting an analyte in a sample.

28. A kit for detecting an analyte of interest in a sample, the kit comprising:
(a) a paper support comprising a capture antibody or antigen coupled to said paper support;
wherein said capture antibody or antigen has affinity for said analyte;
(b) a second antibody for binding said analyte;
wherein said second antibody has affinity for said analyte; and
wherein said second antibody comprises a polymerization catalyst; and
(c) a monomer composition;
wherein said monomer composition comprises a monomer component capable of being polymerized in the presence of said polymerization catalyst.

29. The kit of claim 28, further comprising a basic aqueous solution.

30. The kit of claim 29, wherein the basic aqueous solution is aqueous sodium hydroxide.

31. The kit of claim 28, further comprising instructions for use of said kit for detecting an analyte in a sample.

32. The kit of claim 24, wherein application of a polymerization initiator to said paper support causes at least a portion of said monomer component to form a polymer.

33. The kit of claim 28, wherein said paper support is covalently bound to said capture antibody or antigen.

34. The kit of claim 33, wherein said capture antibody or antigen is covalently bound to said paper support by reacting said capture antibody or antigen with an aldehyde-functionalized paper to produce said paper support.

35. The kit of claim 33, wherein said capture antibody or antigen is covalently bound to said paper support by reacting said capture antibody or antigen with an aldehyde-functionalized paper, followed by blocking unreacted aldehydes to produce said paper support.

36. The kit of claim 33, wherein said unreacted aldehydes are blocked with an aldehyde blocking agent.

37. The kit of claim 24, wherein said analyte is selected from an antigen and an antibody.

38. The kit of claim 24, wherein the polymerization catalyst comprises a photoinitiator.

39. The kit of claim 24, wherein the polymerization catalyst comprises at least a co-initiator.

40. The kit of claim 32, wherein said polymerization initiator is selected form the group consisting of at least one of light, heat, cooling, application of a magnetic field, application of an electrical field, application of electrical current, a chemical reagent and electricity.

41. The kit of claim 40, wherein said polymerization initiator is light.

42. The kit of claim 24, wherein the detecting is colorimetric.

43. The kit of claim 24, wherein said monomer composition further comprises an indicator.

44. The kit of claim 43, wherein said indicator comprises phenolphthalein.

45. The kit of claim 44, wherein said paper support is treated with a base prior to detecting formation of said polymer.

46. The kit of claim 45, wherein detecting formation of said polymer comprises observing a color change mediated by phenolphthalein under basic conditions.

47. The kit of claim 24, wherein said sample comprises blood, plasma, serum, or urine.

48. A method of making a paper support for detecting an analyte, the method comprising:
(a) providing a paper support;
(b) contacting said paper support with an oxidizing agent to produce an aldehyde-functionalized paper support;
(c) contacting said aldehyde-functionalized paper support with said capture antibody/antigen, thereby covalently bonding said capture antibody/antigen to said paper support;
wherein said capture antibody/antigen has affinity for said analyte.
